(19) United States
(12) Patent Application Publication
(10) Pub. No.: US 2004/0138704 A1
(43) Pub. Date: Jul. 15, 2004

(54) Tissue capturing devices

(76) Inventors: Richard A. Gambale, Tyngsboro, MA (US); Michael F. Weiser, Groton, MA (US)

Correspondence Address: KIRKPATRICK & LOCKHART LLP, 75 STATE STREET, BOSTON, MA, 02109-1808, US

(21) Appl. No.: 10/658,619

(22) Filed: Sep. 8, 2003

Related U.S. Application Data
(60) Provisional application No. 60/408,554, filed on Sep. 6, 2002.

(51) Int. Cl. A61B 17/08

(52) U.S. Cl. 606/213

(57) ABSTRACT

The present invention provides tissue capture devices configured to hold tissue in a distorted configuration. The devices may hold precaptured tissue in a distorted configuration or it may change their shape to cause the tissue to become deformed. Some embodiments of the device alter the configuration in areas that remain external to the tissue, while other embodiments change their configuration in areas that are implanted in the tissue. Other embodiments may be mechanically altered to hold the tissue in a distorted shape.

### RELATED DISCLOSURE INFORMATION

The subject matter of the present application is related to the disclosure document filed at the U.S. Patent and Trademark Office on Sep. 7, 2000, and assigned Disclosure Document No. 479569.

### FIELD OF THE INVENTION

The present invention relates to devices and methods for capturing and holding internal tissue portions of the human body.

### BACKGROUND OF THE INVENTION

U.S. Pat. Nos. 5,792,153 and 5,080,663 disclose devices and methods for the endoscopic treatment of gastroesophageal reflux disease (GERD) by suturing together internal tissue locations at the junction of the stomach and esophagus. The devices comprise an endoscopic suturing capsule that is removably attached to the distal end of an endoscope for placing sutures through tissue. The device further comprises a suction chamber into which a tissue portion is aspirated and a reciprocating needle that is advanceable through the tissue to place a suture. The ends of the suture are later drawn outside of the patient and a knot tied to secure the suture in place. By suturing two captured tissue portions together to form a plication and forming series of plications adjacent the Z-line at the junction between the esophagus and stomach, improvements in the symptoms of esophageal reflux have been reported. See Sritharan S. Kadirkamanathan et al., “Antireflux Operations at Flexible Endoscopy Using Endoluminal Stitching Techniques: An Experimental Study”, Gastrointestinal Endoscopy, Vol. 44, No. 3, 1996, pp. 133-143.

The treatment of GERD by the formation of plications at the Z-line may be an effective approach. The presently known methods of applying sutures to create the plications is a cumbersome, lengthy process that requires many separate intubations with the endoscope, which increases risk to the patient of esophageal perforation. It would be advantageous to reduce the number of endoscopic intubations required to form a plication suitable in the treatment of GERD according to the process suggested by Swain and his collaborators. It is an object of the present invention to provide devices and methods used endoscopically for more easily manipulating internal tissue locations and forming plications such as those that are useful in GERD treatment.

### SUMMARY OF THE INVENTION

The present invention provides tissue capturing elements comprising articles and devices deliverable to internal locations in a patient via an endoscope for engaging tissue portions and manipulating those tissues into desired shapes useful in the treatment of various maladies including GERD. The devices and articles may include low profile objects insertable through the working channel of an endoscope or through a catheter or cannula to be delivered to a remote internal tissue location. The low profile devices then may be penetrated through one or more tissue locations and then their shape altered to place the tissue sections in tension, compression or otherwise deform their shape by being constrained together with other captured tissue areas. The tissue capturing devices disclosed herein provide an improvement over the known technique of manipulating tissue by sutures in that the inventive devices can be inserted into the tissue and manipulated to constrain the tissue in a desired shape, all in a single intubation by an endoscope or insertion by a catheter. A single intubation to apply a tissue manipulating device is a great improvement in the art in contrast to the multiple intubations required to insert and secure suture.

The tissue capturing element may comprise a wire-like form having a first, low profile configuration and a second, distorted configuration. The wire-like form is delivered through the endoscope in its low profile configuration inserted around or through a tissue portion. The wire form is then deformed into its second tissue distorting form that serves to hold the tissue, which it engages in a distorted form such as a plication useful in treating GERD.

The wire form may be a straight or curved wire element or a more complicated configuration such as a coil spring. At least a portion of the tissue capturing element should have a tissue engaging portion that either contacts the surface of the tissue and/or penetrates the tissue in order to grasp it and hold it in its distorted form. The tissue capturing element should additionally have at least a portion of its extent being capable of distorting from a first low profile delivery configuration to a second tissue distorting configuration. Examples of tissue distorting configuration may be a straight wire that is changed to form a curve or a small diameter coil spring that changes to form a large diameter coil spring of a much shorter length. When the tissue capturing elements change their form while engaging the tissue, the tissue becomes distorted and the element holds the tissue in that distorted form.

The tissue capturing element should also have a securement mechanism for retaining the element in its tissue distorting form. The securement mechanism may be a mechanical element that holds the wire-like form of a tissue capturing element in a distorted form by mechanically holding it in place. Such a mechanical element may comprise a clasp engageable with the wire form that is malleable. Additionally, the securement mechanism need not be a separate mechanical element but may be a chemical or physical property of the material of the capturing element that causes it to retain a distorted form. For example, a stainless steel capturing element may be configured to have elastic properties so that it can be delivered to the tissue site in a distorted form and then released to elastically return to a second configuration that distorts the tissue that it engages. Alternatively, the securement mechanism may be the shape memory effect possessed by a nitinol alloy material. In this example, a tissue capturing element may be delivered in a low profile form while having a retained memory shape that is distorted to a different configuration. Therefore, after the nitinol element is delivered into the body, the increased temperature presented by the body will trigger the transformation of the nitinol material to the retained shape memory configuration thereby distorting the tissue engaged by the element and holding it in place.

It is an object of the present invention to provide tissue capture devices that can be delivered into internal tissue to hold the tissue in a distorted form by their implanted configuration or by a change in configuration after implantation.

It is another object of the invention to provide tissue capture devices that alter their configuration in areas that are implanted in the tissue or in areas that are external to the tissue or that modified on their external surfaces to remain implanted within the tissue.

It is another object of the invention to provide a method of capturing internal tissue areas in a distorted form using a tissue capture device.

### BRIEF DESCRIPTION OF THE DRAWINGS

The foregoing and other objects and advantages of the invention will be appreciated more fully from the following further description thereof, with reference to the accompanying diagrammatic drawings wherein:

FIGS. 1-3 show successive steps in the operation of a prior art single stitch sewing device;

FIG. 4 is a diagrammatic side view of a tissue apposition device mounted to an endoscope;

FIG. 5 is a diagrammatic side view of a tissue apposition device mounted to an endoscope;

FIGS. 6A-6B are isometric views of a multiple suction port apposition device in various stages of operation;

FIGS. 7A-7C are views of a multiple endoscopic band ligator;

FIGS. 8A-11B are side sectional views of tissue capture devices that transform their shape in areas implanted within the tissue after implantation;

FIGS. 12A-12B show the implantation of a tissue capture device that changes its configuration after implantation;

FIGS. 13A-14B are side sectional views of tissue capture devices implanted in tissue that changed their configuration in areas that are external to the captured tissue;

FIGS. 15A-15B are side sectional views of a tissue capture device placed in tissue and being secured by a capture element.

FIGS. 16A-16B are side sectional views of a tissue capture device placed through tissue and experiencing a removal of a coating to expose a roughened surface that captures the tissue;

FIGS. 17A-17B are side sectional views of a tissue capture device implanted through tissue then joined together subsequent to implantation.

FIGS. 18A-18B show a tissue capture device comprising a straightened coil spring that is permitted to return to its coiled form during delivery;

FIGS. 19A-19C show tissue capture devices that are implanted directly into tissue without undergoing a shape change;

FIGS. 20-21 are side sectional views of tissue capture devices implanted through tissue then secured externally;

FIG. 22 shows a side sectional view of a tissue implant device comprising a reverse wound spring;

FIGS. 23A-24F show a tissue capture device comprising a dart and flexible tether and its delivery to tissue;

FIG. 25 is a side sectional view of the tissue capture device configured as a dart with flexible tether implanted in tissue and secured;

FIGS. 26A-26D show side sectional views of a tissue capture device delivered through tissue portions captured by ligating bands;

FIGS. 27A-27D are side sectional views of a tissue capture device that is implanted into non-captured tissue and later transforms to capture and deform the tissue;

FIGS. 28A-28D show a tissue capture device comprising two helical springs joined by a super elastic hypo tube;

FIGS. 29A-29J show a tissue capture device configured as a tweezer temporarily capturing tissue to deliver a suture.

### DESCRIPTION OF THE ILLUSTRATIVE EMBODIMENTS

The present invention provides devices for holding tissue that is an alternative to conventional flexible suture material. The devices have at least a semi-rigid form after implantation into the tissue that is capable of maintaining a definite shape useful in holding the tissue in a deformed configuration. The devices may hold a single tissue area in a distorted configuration or may be used to hold two or more tissue areas in a distorted configuration and in close proximity to each other. Tissue collected into a distorted configuration appears as a mound of tissue and will henceforth be referred to as a tissue mound in this application.

The embodiments disclosed herein may be segregated into several categories. Several devices are used with formed tissue mounds that are collected and temporarily held in a distorted shape prior to application of the device. After the device is inserted it holds the tissue in the deformed configuration. Other embodiments may be applied to a tissue area that is not held in a deformed shape because the tissue deforms when the inserted device deforms into its alternate configuration.

Several embodiments of the devices employed into tissue pre-collected into a mound shape may be placed directly into the tissue mound to retain the distorted tissue shape without the device undergoing a configuration change of the device. Other embodiments are placed into the formed tissue mound and undergo a change in configuration only in areas of the device that remain external to the tissue mound after insertion in order to maintain the tissue mound shape. Still other embodiments are placed into the formed tissue mound and undergo a configuration change in areas of the device that are implanted within the tissue in order to maintain the distorted mound shape in the tissue.

The tissue may be collected into a deformed, mound shape by a separate instrument such as forceps or by a specialized tissue capturing device such as the endoscopic suturing capsule disclosed in U.S. Pat. No. 5,792,153 or in a multiple suction port device to capture a plurality of tissue mound simultaneously such as that disclosed in U.S. patent application Ser. No. 10/220,379. The entirety of both referenced documents are incorporated by reference in their entirety in this application. To provide a complete understanding of how the tissue capturing devices of the present invention may be employed into temporarily captured mound of tissue, a description of the operation of the prior art tissue apposition devices is provided. Use device can be used to capture tissues into formed mounds and then facilitate insertion of the capture devices, rather than a suture, to hold the tissue in position.

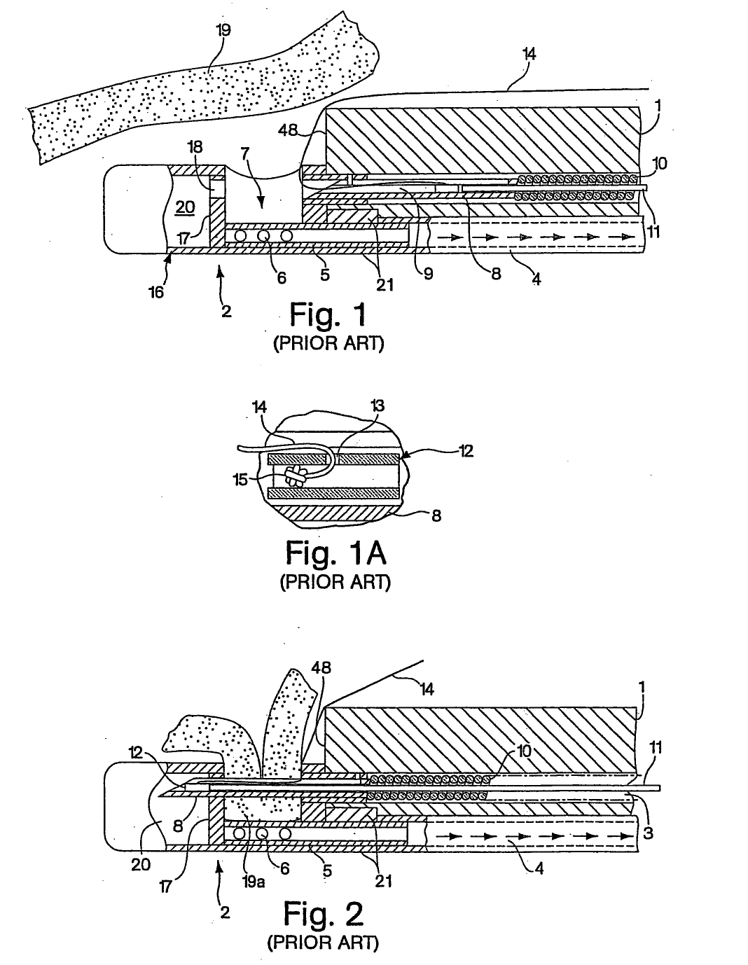

FIGS. 1-3 depict a prior art endoscopic suturing device disclosed in U.S. Pat. No. 5,792,153. FIG. 1 shows the distal end of a flexible endoscope 1, on which a sewing device 2 is attached. The endoscope is provided with a viewing channel, which is not shown, but which terminates at a lens on the distal face of the endoscope. The endoscope is further provided with a biopsy or working channel 3, and a suction channel 4 the proximal end of which is connected to a source of vacuum (not shown). The suction channel 4 may comprise a separate tube that runs along the exterior of the endoscope, rather than an internal lumen as shown. The sewing device 2 has a tube 5, which communicates with the suction pipe 4 and has a plurality of perforations 6 therein. These perforations communicate with an upwardly open vacuum chamber 7 formed in the sewing device.

A hollow needle 8 is mounted in the biopsy channel 3, with its beveled tip extending into the sewing device. The needle has a channel 9 extending therethrough. A flexible, wire-wound cable 10 has its forward end attached to the rear of the needle 8, and a center wire 11 runs within the cable 10, along the entire length thereof, and is longitudinally movable with respect thereto. The diameter of the wire 11 is such that it is longitudinally movable within the channel 9 and, in the position shown in FIG. 1, the forward end portion of the wire 11 extends into the rear end portion of the channel 9. A thread carrier in the form of a tag 12 is slidably and releasably mounted in the channel 9. The tag is shown in detail in FIG. 1A. The tag is hollow and has an aperture 13 extending through the sidewall thereof. As can also be seen in FIG. 1, one end of a thread 14 is secured to the tag by passing it through the aperture 13 and tying in the end of a knot 15 of sufficient size to prevent the thread escaping from the tag. The tag may be made from a relatively rigid material such as stainless steel.

At the distal end of the sewing device is defined a hollow head portion 16 defining a chamber 20 therein. Between the chamber 20 and the cavity 7 is a wall 17, in which an aperture 18 is formed. The aperture 18 has a diameter that is marginally greater than the external diameter of the needle 8, and is aligned therewith. The clearance between the needle 8 and the aperture 18 must be sufficiently small to prevent tissue being forced through the aperture and causing the needle to jam. Finally, FIG. 1 shows a portion of the patient's tissue 19, in which a stitch is to be formed.

In operation, suction is applied to the suction pipe 4, and thence, via the perforations 6 in the tube 5 to the cavity 7. This sucks into the cavity a U-shaped portion 19a of the tissue 19, as shown in FIG. 2. The hollow needle 8 is pushed through the U-shaped tissue portion 19a by extending distally the wire-wound cable 10 and associated needle 8. After full advancement of the needle through both folds of the U-shaped tissue portion, the tip potion of the needle 8 is distal to the wall 17 and within the chamber 20 in the hollow head portion 16. Distal movement of wire 11, slidably received within the wound cable 10, pushes the tag 12 out of the channel 9 and into the chamber 20 where it rotates out of alignment with aperture 18 to become captured in the chamber.

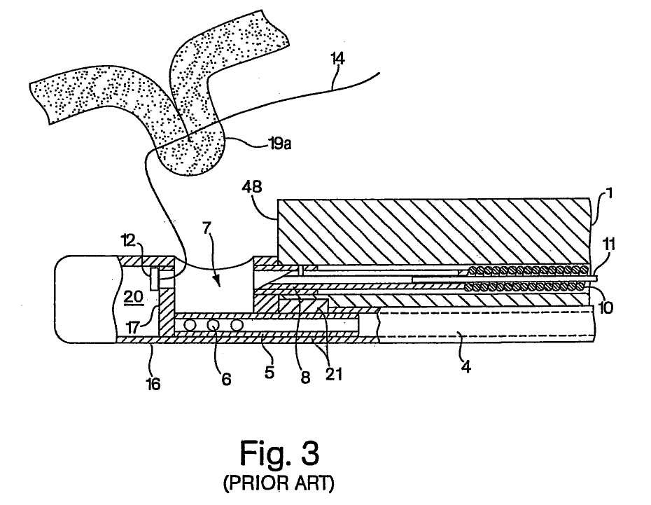

The wire 11 is then withdrawn proximally, followed by proximal withdrawal of the cable 10, to withdraw the needle 8 from the tissue portion 19a. The suction is then discontinued allowing the U-shaped tissue portion 19a to be released from the cavity 7. As shown in FIG. 3, the released tissue is left with a suture thread 14 passing through the two layers of tissue that form the U-shaped fold 19a. One end of the suture is joined to the tag 12 that remains captured in the chamber 20 and the other end of the suture extends through the patient's esophagus and out of the mouth. Finally, the endoscope and dewing device are withdrawn from the patient. In so doing, the thread 14 is pulled partially through the tissue portion 19a, as the captured tag 12 is withdrawn proximally and brought outside the patient. With both ends of the thread 14 outside of the patient, the thread can be knotted and the knot endoscopically pushed down to the suture site and severed by an endoscopic knot pusher such as that disclosed in U.S. Pat. No. 6,010,515 (Swain et al).

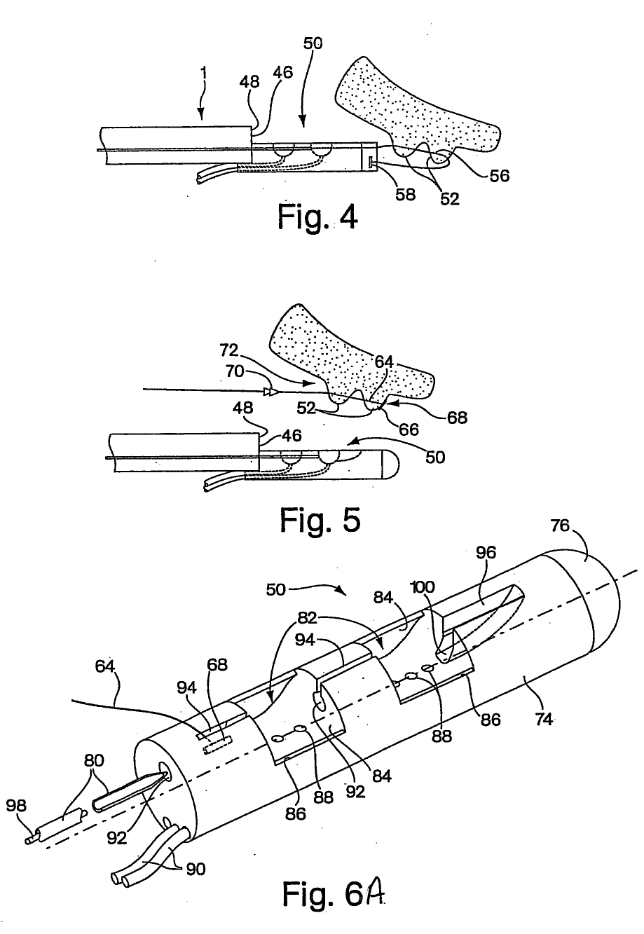

For certain treatments, capturing multiple tissue portions, gathering and holding them together may be desirable. FIGS. 4-5 illustrate the operation of a multiple suction port apposition device 50 as disclosed in co-pending U.S. application Ser. No. 10/220,379. The device can capture multiple tissue portions 52 simultaneously for application of a tissue securing device, such as a suture, tag or staple. The device may be modified to deliver the tissue securing devices of the present invention. Securing two tissue portions 52 in the same number of steps that the prior art device requires to secure a single tissue portion doubles efficiency, reducing the total number of endoscopic intubations required to complete the procedure and reducing the time needed to complete the procedure. Though dual suction port embodiments are discussed for illustration purposes, it should be understood that the multiple port device also could be configured to have three or more suction ports.

The prior art dual suction port tissue apposition device shown in FIG. 4 passes through both tissue portions a suture 56 with a tag 58 capturable in the end cap 60 of the sewing capsule 62, in similar fashion to the prior art device discussed above. The dual suction port tissue apposition device shown in FIG. 5 passes through both tissue portions a suture 64 having a permanent tag 66 at its end. In this embodiment, the permanent tag is not captured by the suturing device to later provide a lead for tying a surgical knot. Rather, the permanent tag remains in the body, anchored on the through side 68 of the distal tissue portion. The tissue portions may then secured tightly together, not by a surgical knot, but by a frictionally engageable two piece suture lock device 70 advanced along the single suture lead 64 to abut the proximal side 72 of the tissue portion.

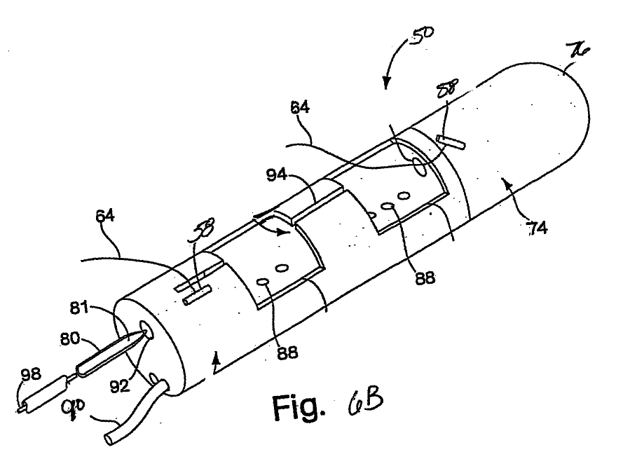

In one embodiment of the multiple suction port device, the multiple suction ports are defined in line on the sewing device, along a common longitudinal axis that is parallel to the longitudinal axis of the device. An isometric view of an in-line dual suction port endoscopic tissue apposition device 50 is shown in FIG. 6. In FIG. 6, a slotted and beveled hypodermic suturing needle 80 is in the fully retracted position, with suture tag 68 not yet loaded, and the capsule ready to receive tissue. The sewing device 50 is characterized by a tubular body or capsule 74 that is machined from metal or injection molded from a rigid polymer material. The body may be formed with an atraumatic distal tip 76 to avoid injury to the walls of a body lumen through which the device is delivered.

A plurality of suction ports 86 are formed into the body along its length. Suction ports 86 are large openings defined through the capsule 74, and open to one or more vacuum chambers 82. The chambers are defined in the capsule by surfaces forming sidewalls 84. Communication of the suction ports with the vacuum chambers 82 permits vacuum to reach tissue that is adjacent to the ports to accomplish capture of tissue portions 52 into the chamber. Any number of suction ports can be formed on the capsule body. However, two suction port devices are shown here as illustrative examples because often in the treatment of GERD, a series of two tissue mounds joined together are formed along the stomach wall, below the Z-line. Though more ports and chambers can be formed on the body, the extra body length they would require in the in-line embodiment could potentially present difficulty during navigation of the rigid body through the curves of a natural body lumen.

Tissue portions are drawn into the suction ports and into the vacuum chambers by suction introduced to the chambers through air passages 88. The air passages are open to independent internal channels in the body that are joined to vacuum lines 90. The vacuum lines extend from the proximal end of the capsule body, external to the endoscope, to the proximal end of the scope. Outside the patient, the vacuum lines can be joined to a portable or institutional vacuum source (not shown). A control valve may be inserted in-line near the proximal end of the tubes for selective control of the vacuum by the user. The air passages of all cambers may be joined and controlled by a single vacuum line. Alternatively, as shown in FIG. 6, separate vacuum lines may be used to supply suction to the air passages of different vacuum chambers. Use of separate vacuum lines permits independent control of suction provided to the several chambers by the use of separate control valves for each vacuum tube at their proximal ends.

Independent vacuum supply to the air passages of each chamber not only helps to ensure adequate vacuum pressure to each chamber, but also permits sequential suctioning of tissue into the chambers. When tissue is collected into both chambers simultaneously, the distal chamber is blocked from the viewing lens 48 on the distal face 46 of the endoscope 1, as shown in FIG. 5. Therefore, the physician is unable to visually determine whether tissue has been adequately collected into the vacuum chamber so that the needle 80 can be safely advanced through. By applying vacuum first to the distal chamber, tissue collection into that chamber can be visually verified before the view is blocked by tissue entering the proximal chamber. Next, vacuum can be applied to the proximal chamber to capture tissue so that tissue is collected in both chambers simultaneously and held in readiness for penetration by the suture needle (or staple) through both tissue portions with one stroke. However, even with independent vacuum lines, it is possible, and may be desirable to apply a vacuum to all chambers simultaneously.

The needle 80 is longitudinally slidable through the capsule body 50, as in the prior art devices. In the in-line dual chamber embodiment shown in FIG. 6A, a tunnel-like needle track 92 extends longitudinally through solid portions in the upper half of the body, not otherwise defined by the vacuum chambers. From the needle track, a thin suture channel 94 extends upwardly through the top surface of the capsule body to provide a space through which the suture lead 64 may pass as the suture tag 68 is advanced by the needle through the needle track 92. The channel 94 is only a sufficient width to permit the suture to pass but is too small to permit passage of the larger needle or suture tag 68. The small dimension of the channel helps maintain the needle and suture tag with in the needle track until they are extended distal to the most distal chamber. An enlarged exit channel 96 extends upwardly from the needle track along the body a short distance distally from the distal chamber 82. The enlarged channel facilitates exit of the suture tag 68 from the body, to follow the released tissue to which it has been attached after being ejected from the extended needle 80 by pusher wire 98. Additionally, a ramp 100 may be formed in the bottom surface of the needle track along the length of the exit channel 96. Extending upwardly as it extends distally, the ramp 100 helps guide an ejected tag up and out from the exit channel and away from the capsule body. A detailed isometric view of the dual suction chamber device of FIG. 4 in which the tag 58 is captured in the distal end 76 of the device is shown in FIG. 6B.

FIG. 6C shows another embodiment of the multiple port tissue apposition device in which the suction ports are arranged side-by-side rather than longitudinally in line as were the above-described embodiments. The suturing capsule 200 has a tissue capture mechanism comprising two or more suction ports 202 that arranged side-by-side, angularly offset but substantially aligned with each other longitudinally (referring to the longitudinal axis of the capsule and endoscope). The suction ports 202 define openings into the capsule 200 and are separated by partition 204. As with the previous embodiments, suction ports 202 open to a vacuum chamber 206 defined by sidewalls 208 inside the capsule 200. As with the above embodiments, vacuum is created in the vacuum chambers through negative pressure introduced by air passages 88 (not shown) to cause tissue to be drawn into the vacuum chambers through suction ports 202. The air passages are in communication with vacuum channel 234 formed through the capsule body and joinable to a vacuum channel 4 of the endoscope or an independent vacuum line.

As tissue is drawn into the suction ports 202 under vacuum, the partition 204 causes the tissue to be separated into two distinct mounds or portions into which tissue securement means such as sutures may be driven as is described below. The suction ports 202 may be in communication with a single, common vacuum chamber 206 (as shown in FIG. 6C) or each suction port may open to independent, dedicated vacuum chambers that can be separately evacuated. Separate vacuum chambers would further be defined by a sidewall extending from partition 204 into the vacuum chamber 206.

An alternative device for capturing tissue portions by suction may be configured similar to an endoscopic band ligator such as those disclosed in U.S. Pat. No. 4,735,194 (Stiegmann) or in U.S. provisional patent application No. 60/408,555. The entirety of those documents are incorporated by reference in their entirety.

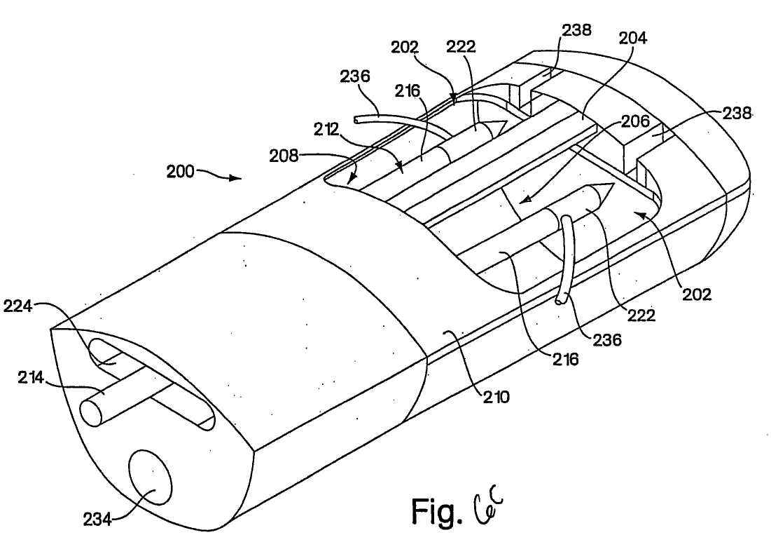

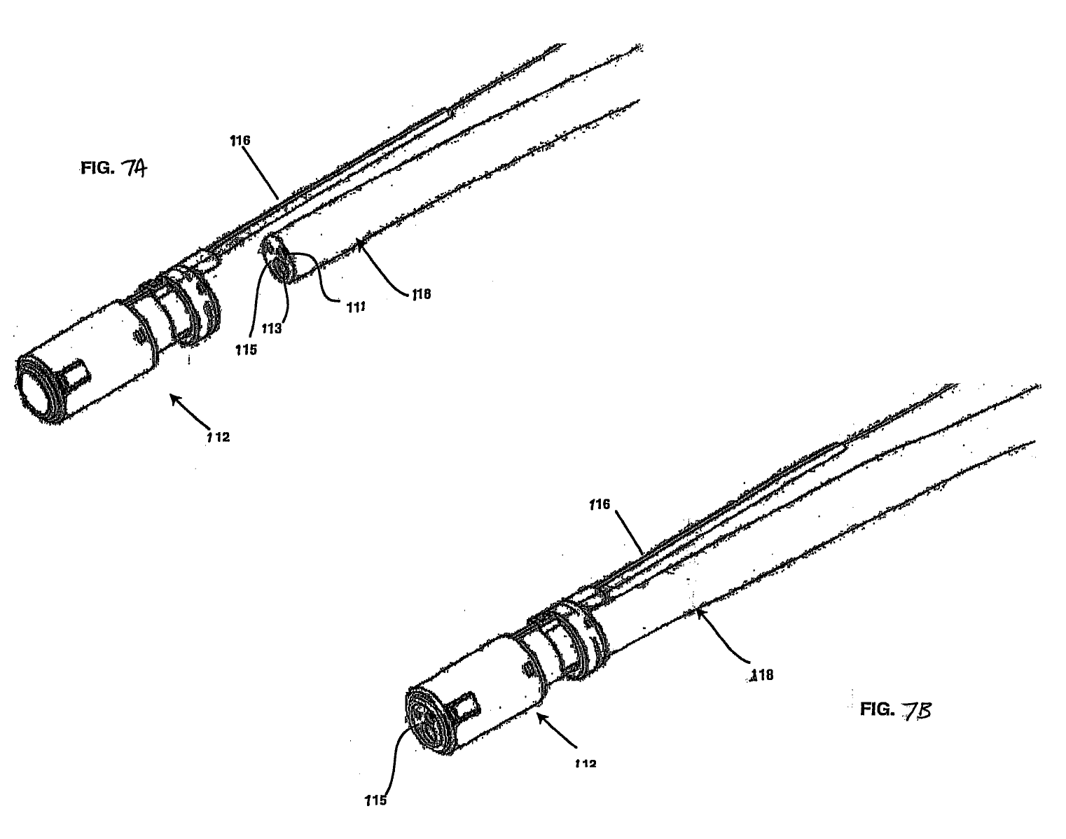

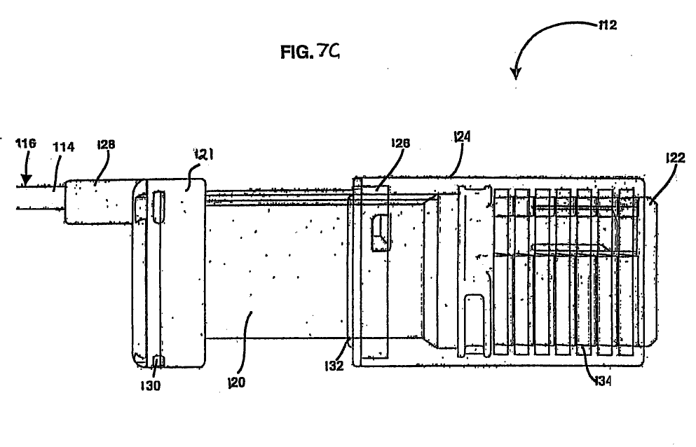

The ligator device of the '555 application is slidably mounted onto a distal end of an endoscope 18 and is frictionally retained on the endoscope as is shown in FIGS. 7A and 7B. The ligator 12 is backloaded onto the distal end 18 of the scope and slid proximally so that the distal end of the distal portion is substantially flush with the distal face 15 of the scope. A sheath 16 containing control wires and connected to the distal portion, extends parallel to the endoscope shaft, proximally to a control handle. When the device is navigated to a tissue treatment site, the tubes are in a retracted position, such that the band driver 24 and band carrier 22 are positioned proximally on the static sleeve 20. In this position, the distal portion 12 does not interfere with the peripheral view through the viewing lens 11 on the distal face 15 of the endoscope (FIGS. 7A & 7B).

When the tissue treatment site has been reached, the band driver 24 and band carrier 22 together are slid distally relative to static sleeve 20 to the position shown in FIG. 7C. By their distal movement on the static sleeve, the band carrier 22 and band driver 24 together are extended beyond the distal face of the endoscope. The cylindrical interior of the band carrier creates a vacuum chamber, closed at its proximal end by the endoscope distal face 15 and open at its distal end to receive tissue. Band carrier 22 and driver 24 are preferably made from transparent polymer materials to minimize interference with peripheral viewing through the endoscope when they are advanced beyond the distal face 15. Tissue is aspirated into the vacuum chamber when suction is applied through the vacuum port 13 on the distal face of the endoscope. With the tissue aspirated into the suction chamber, the band driver 24 is then slid distally relative to the band carrier 22 to push a band 34 from the band carrier and onto the tissue.

FIG. 8A shows a nitinol capture device 302 having a V-shape with two prongs 304 each inserted into the top of a separate tissue mound 306 that had been previously manipulated into the mound shape by separate means such as one of the devices discussed above. As shown in FIG. 8B, the nitinol capture device is preformed so that upon exposure to the elevated temperature of surrounding body tissue, the prongs 306 that extend into the tissue undergo a configuration change due to the shape memory effect of the nitinol. In this example, the nitinol is preconditioned to form zigzags 308 through each prong 304 extending through a tissue mound 306. Transformation to a sinusoidal or zigzag shape as shown by 308 in FIG. 8B serves to hold each prong 304 in the tissue bound 306 so that it is not easily removed through the mound. The V-shape of the capture device 302 is maintained, despite the shape memory change of the nitinol material in order to maintain the captured tissue mounds 306 held together in close proximity as is shown in the figures. It is contemplated that the capture device could be delivered endoscopically in a multiple suction port tissue apposition device such as that shown in FIG. 6. The tissue capture mechanism could be arranged in the suction port such that each of the prongs 304 upwardly and outwardly in each of the ports so as tissue is sucked into the port, the prongs will be driven into each tissue mound that is formed and captured by the device.

In FIGS. 9A and 9B as shown another nitinol capture device 310 that operates in a similar fashion to that shown in FIGS. 8A and 8B. As with the earlier embodiment, the device is configured to have a V-shape with each prong 312 of the V inserted into an adjacent pre-captured mound of tissue 306 while in a relatively straight configuration. After exposure to the increased temperature of tissue surrounding the prong 312, the nitinol material undergoes a shape transformation back to a pre-trained configuration that corresponds to the arrangement of molecules of the material at the higher temperature. In the case of the capture device 310 as shown in FIG. 9B, each prong 312 changes shape to have a barb 314 at its free end that serves to anchor the device into the tissue. Portions of the device that remain external to the tissue mounds do not undergo a shape change. It is expected that the nitinol capture device 310 will be implanted in the same manner as disclosed above for the embodiment of FIGS. 8A and 8B.

Another embodiment of a tissue capture device deployed into pre-captured tissue mounds is shown in FIGS. 10A and 10B. The capture device 318 comprises a helical spring that is inserted through two adjacent tissue mounds 306 that have been pre-captured. After the spring is inserted through the tissue mounds 306, it transforms into a increased diameter shorter length configuration that secures it in the tissue mounds and draws the tissue mounds close together. The spring type capture device may transform from a low profile to a large profile by either the mechanism of shape memory if formed from a nitinol material, or by resilient expansion inherent in the material such as stainless steel. A nitinol spring may be threaded directly into the sides of captured tissue mounds 306 while they are captured by a longitudinally arranged multiple port suction device such as that shown in FIG. 6. In the case of a resiliently expandable spring steel, the spring type capture device should be maintained in a rigid delivery tube to confine its profile during insertion through the tissue portions 306. The rigid insertion tube also can be advanced longitudinally through a multiple suction portion apposition device such as that shown in FIG. 6. Once inserted through the tissue mounds, the spring may be held in position by an inner push rod while the rigid tube is withdrawn proximally from the tissue, allowing the spring to expand as it is unsheathed.

FIG. 11A shows another capture device, similar to the embodiments of FIGS. 8B and 9B, but incorporating an umbrella anchor 324 at the free end of each prong 322. The capture device is inserted into the pre-formed tissue mounds 306 with the prongs 322 in a straight configuration as shown in FIG. 11A. After implantation, the prongs 322 have expanded at their free ends. Small umbrella anchors 324 to hold the device in the tissue. The mechanism for expansion of the umbrella anchors may be shape memory effect if the device is formed from nitinol or may be resilient expansion if the device is formed from stainless steel. If formed from stainless steel, it is expected that a confining sheath will be placed over the umbrella anchors 324 during insertion into the tissue to maintain them in a low profile. After implantation, the sheath may be removed from the device to permit resilient expansion of the anchors. The device may be delivered to the captured tissue mounds 306 by a multiple chamber suction device such as shown in FIG. 6, each prong of the device may be delivered separately by an axially oriented suction device such as the ligator device shown in FIGS. 7A-7C.

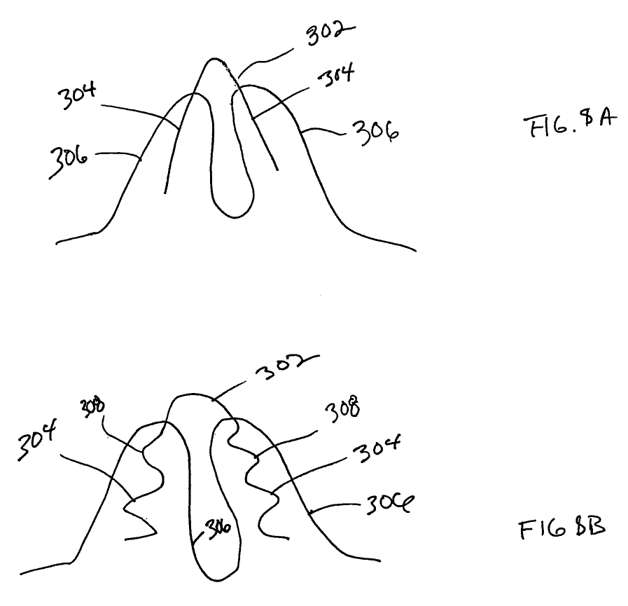

FIGS. 12A-12D illustrate the delivery of another embodiment a nitinol tissue capture device. The device 340, is placed into a pre-captured tissue mound 306 and after exposure to the elevated temperature of the tissue, changes its configuration in areas that are embedded in the tissue to serve to hold the tissue in the mound shape. The device 340 resembles a staple, having two prongs 342 arranged in parallel and joined to a perpendicular cross member 344. The cross member is configured to transform to a compressed configuration when exposed to the elevated temperature of the tissue by virtue of the shape memory effect of the nitinol material from which it is formed.

The device 340 may be placed in a single mound 306 of pre-captured tissue, as is shown in FIG. 12A. To pre-capture the mount of tissue 306, an endoscopic ligator device 112 such as that discussed above in connection with FIGS. 7A-7C may be employed. As shown in FIG. 12A, an endoscope 118 carrying a ligator 112 is navigated to a tissue location and a mount of tissue 306 aspirated into the suction chamber of the ligator. A ligating band 134 is advanced distally from the device to surround the aspirated tissue mound 306 as described above in the operation of the device. Next, the device 340 may be advanced distally into the top of the tissue mound 306. The device may be advanced by a slidable pusher 346 extending through the working channel of the endoscope 118 and having an device engaging member 348 at its distal end. The device is advanced so that the prongs 342 become embedded into the tissue. The cross member 344 becomes flush with the top of the tissue mound where it becomes slightly embedded when the device is fully seated (FIG. 12B).

As shown in FIG. 12C, after the device is placed in the tissue mound, the endoscope and ligating device may be removed from the tissue location. The ligating band 134 holds the tissue mound in the desired shape while the cross member 344 undergoes its shape memory transformation to a compacted, sinusoidal form. The compact sinusoidal form of the cross member 344 tends to pull the prongs 342 closer together which, after implantation, serves to pinch the tissue in a gathered form that retains the desired mound shape. Also as shown in FIG. 12C, the prongs 342 may be configured to have barbs 349 project slightly outward to hold the device 340 in the tissue. After the device has had sufficient time to transform its shape, the ligating band 134 may be removed from the tissue mound, as it will no longer be needed to retain the distorted shape of the tissue. The band may be removed by cutting or it may be formed from a degradable material that disintegrates a suitable time after implantation in the body and after the device 340 has transformed to its second profile, as is shown in FIG. 12D.

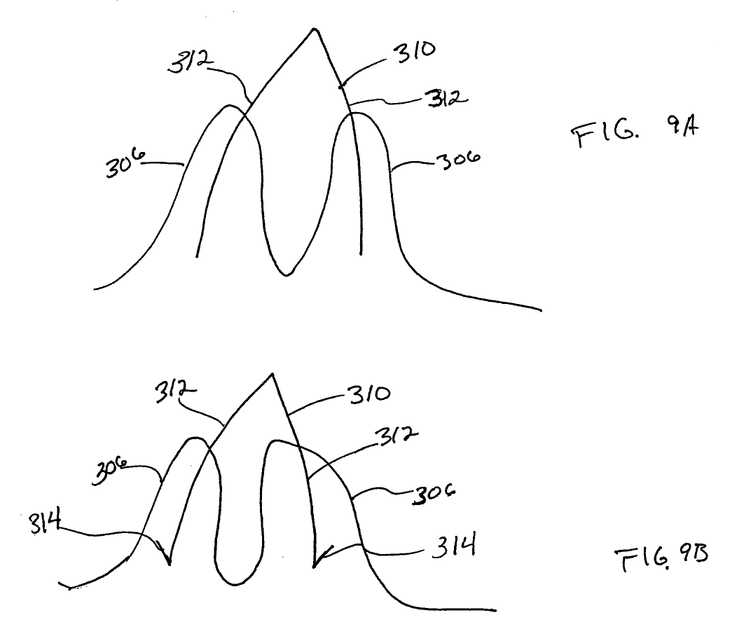

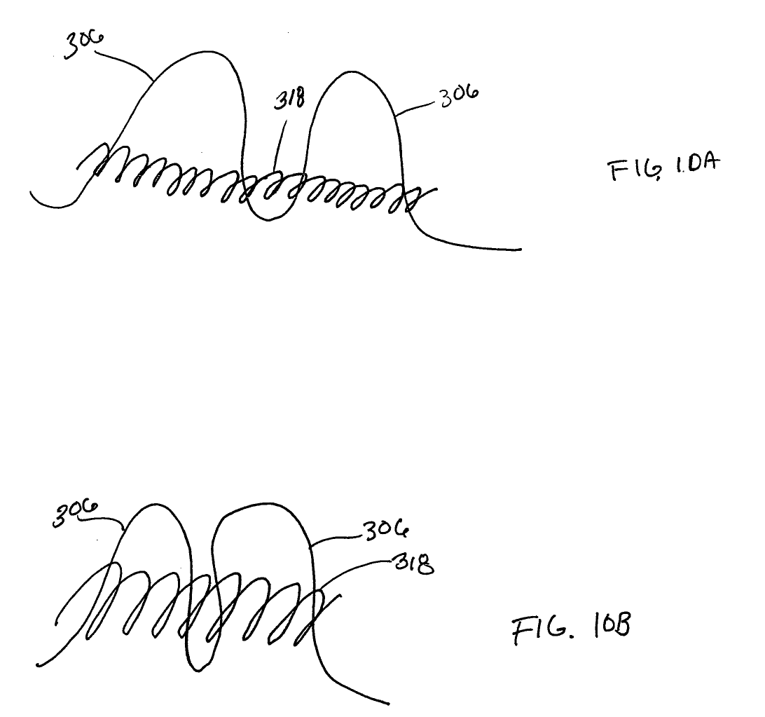

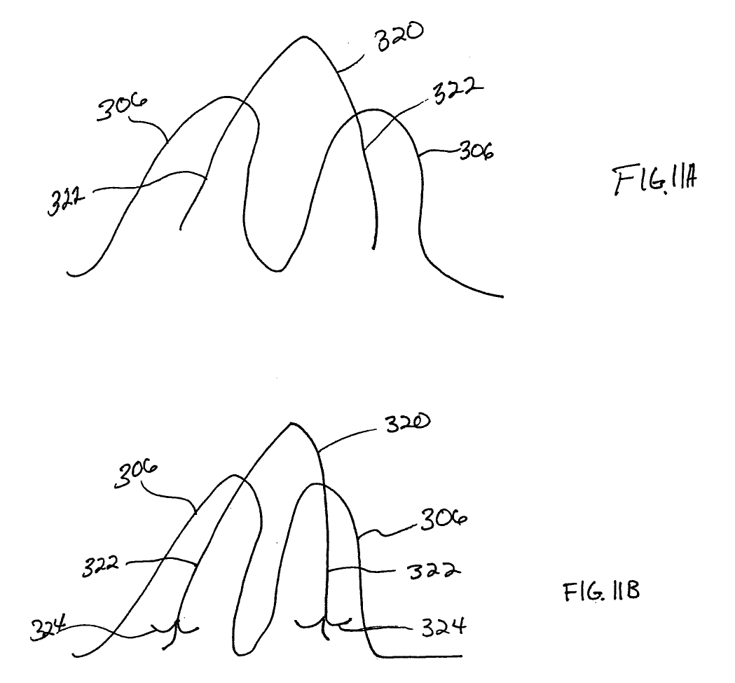

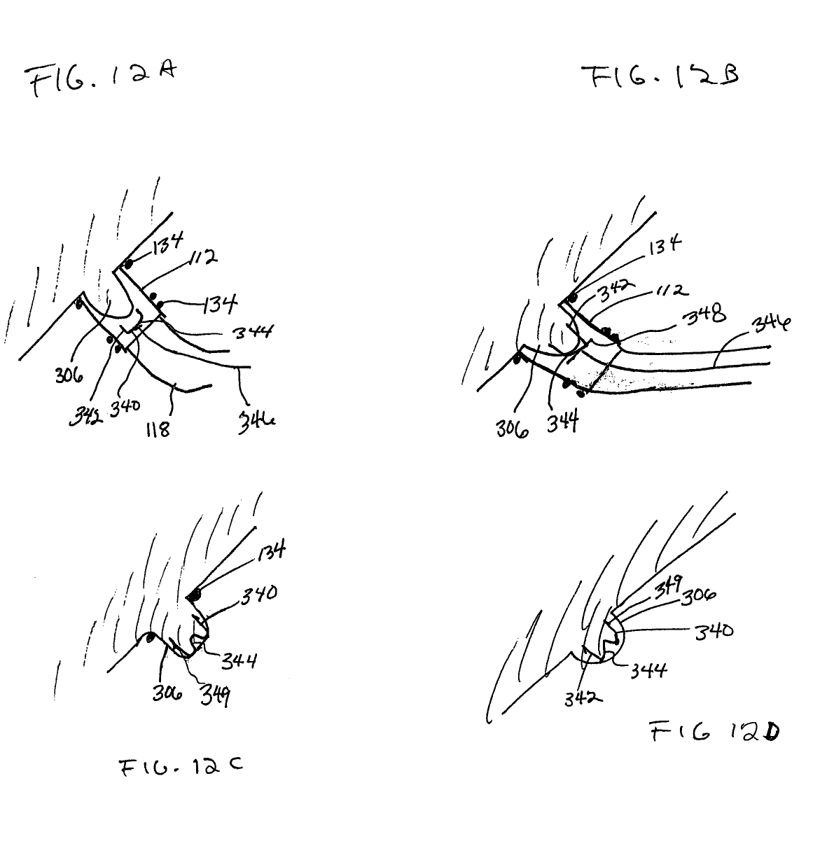

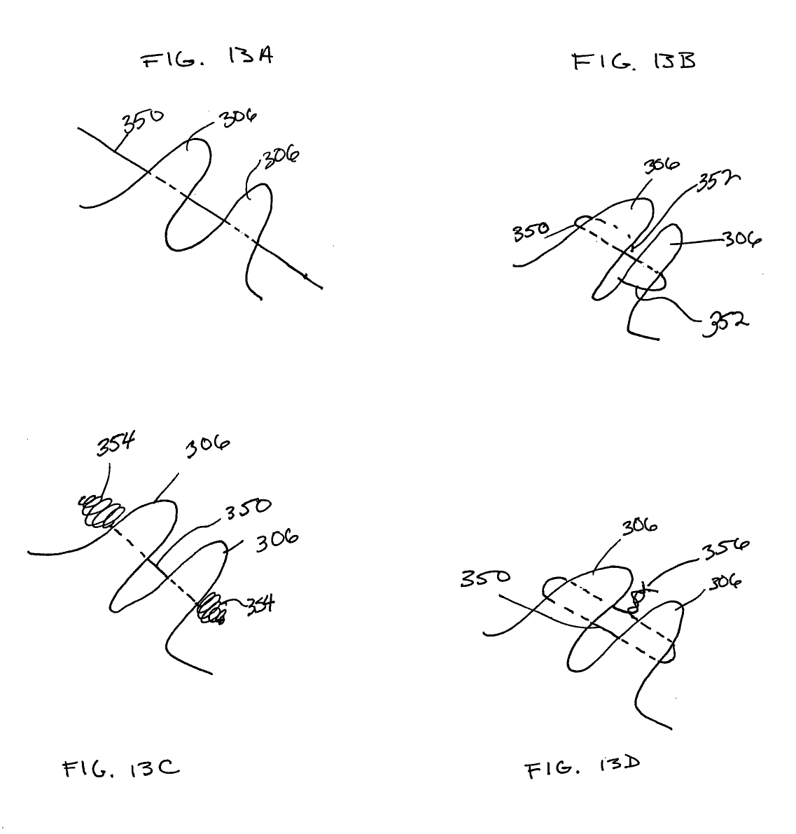

In another group of embodiments, the capture device is configured to be inserted into a pre-deformed tissue and retain it in that shape by reforming its shape only in areas that remain external to the tissue mounds. FIG. 13A shows an device delivered through two adjacent collected mounts of tissue 306 prior to any transformation of the device to a different configuration and profile. FIGS. 13B-13D show various second configurations of the nitinol device that may be employed to keep the device in the tissue mounts and the mounds close together. In each of the embodiments of FIGS. 13B-13D, the nitinol device undergoes a transformation to its second configuration only in areas of the device that remain outside the tissue. In FIG. 13B, the device 350 is configured to have end portions that undergo a shape memory transformation to U-shaped curves 352 that are sized to approximately wrap around one side of each of the captured tissue mounds 306. The curved ends 352 of the device 350 serve to hold the device in place relative to the tissue mounds 306 and hold the mounds in close proximity relative to each other.

In FIG. 13C, the device 350 is configured to have free ends that are configured to undergo a shape memory transformation causing them to reconfigure as helical coils 354. The coiled ends are larger profile than the original straight linear device 350 that was inserted through the tissue mounds 306, therefore, they cannot pass through the hole in the tissue created by the insertion of the device in its straight configuration. The coiled ends 354 on either side of the tissue mounds 306 thus serve to hold the device 350 in position relative to the tissue and serve to hold the tissue mounds 306 in close proximity to each other.

FIG. 13D shows another shape memory transformation possibility where the free ends of the device 350 are configured to undergo a shape change transformation in which they wrap around a side of each mound 306 and become engaged with each other in a twisted form 356.

FIGS. 14A and 14B show another embodiment of a tissue capture device 360 that operates to bring a plurality of tissue mounds together by a shape transformation in areas of the device that remain external to the tissue after implantation. The tissue capture device 360 comprises two or more prongs 366 joined by a deformable bridge 364 to define a generally U-shaped implant. Prior to and during implantation, the bridge 364 is maintained in a relatively straight configuration by a removable brace 362 so that the prongs 366 remain spaced apart in a U-shaped configuration that is easy to insert into pre-captured tissue mounds 306 (FIG. 14A). The bridge 364 is preferably formed from a different material from that of the prongs 366 and has a predefined and unrestrained configuration that is more compact so as to draw the ends of the prongs 366 closer together to draw captured tissue portions together after release of the device. As shown in FIG. 14B, the bridge 364 may transform into a loop or coil to reduce the length of the bridge and draw the prongs 366 closer. The inherent predefined shape of the bridge may be caused by resilient spring tension in the case of the stainless steel bridge member or may be a preformed shape memory configuration if formed from nitinol. To temporarily hold the bridge in a straight configuration during implantation, the bridge is held in a straight form and has molded around it a biodegradable polymer of sufficient strength to maintain the bridge in the straight configuration. After some exposure to the interior of the human body, the brace 362 degrades and ultimately releases the bridge section to reform into its unrestrained configuration as shown in FIG. 14B.

FIGS. 15A and 15B show another tissue capture device 370 implantable into a plurality of tissue mounds 306 and deformable on its external surfaces to bring the tissue mounds in close proximity. The device comprises a pair of tissue prongs 372 arranged substantially parallel to each other and linked together at their proximal ends by an adjuster 374. The adjuster 374 is slidable along both of the prongs such that sliding in the distal directions serves to bring the prongs together to a fixed distance that is in close proximity to one another. In use, the device 370- is delivered to a tissue location in which two tissue mounds of pre-captured to delivery by a device such as that shown in FIG. 6. The device 370 is inserted such that each of the prongs 372 is inserted into the top of a tissue mound 306, as shown in FIG. 15A. After implantation, the adjuster 374 is advanced distally over the ends of the prongs 372 so that the prongs are brought together along with the tissue portions 306 into which they are then inserted, as shown in FIG. 15B.

Other embodiments of the tissue capture device inserted into pre-captured mounds of tissue retain their shape after being inserted into the tissue, yet are still capable of holding the tissue in place. FIGS. 16A and 16B show a device 380 having a roughened outer surface 382 that is temporarily covered during insertion into the tissue by a dissolvable polymer 384. The device 380 may have any shape capable of penetrating the captured tissue mounds 306, such as the linear piercing shape shown in FIGS. 16A and 16B. After insertion through both tissue mounds in tended to be captured together, the biodegradable substance 384 will dissolve away after coming into contact with the tissue. Left exposed will be the roughened surface 382 that will grip the tissue mounds and hold them together, as well as hold the device in place within the tissue. The roughened surface 382 may be comprised of small bumps where barbs are formed on a metallic device of any cross-sectional shape. The small projections of the roughened surface engage the tissue to prevent movement of the device. The degradable coating may be any material that is easily applied to the device prior to implantation and is capable of degrading quickly in the presence of the environment of internal body tissue. Poly L lactite polymers are a possible coating material that can be used to cover the device and smooth over the roughened surface to facilitate initial insertion through the tissue mounds 306. The device 380 may easily be delivered by an endoscopic tissue apposition device such as that shown in FIG. 6, which is capable of capturing two mounds of tissue and advancing a longitudinal element through the captured tissue mounds.

FIGS. 17A and 17B show another embodiment of a tissue capture device 390 that is inserted into captured tissue mounds 306 into separate components that are later joined together and after insertion to pull the tissue mounds 306 in close proximity. The device 390 may comprise a helical spring that is implanted into the tissue by rotating such that the helical winding is screwed into the tissue. The individual coils 392 serve to capture the device 390 and the tissue mound 306. As mentioned above, a second coil device 390 is placed in an adjacent tissue mound during the insertion process. The implantation process may be carried out using a device similar to that shown in FIG. 6 in which two tissue mounds 306 are captured simultaneously. The coil spring may then be delivered longitudinally through the mounds along the longitudinal axis of the device, such as through the working channel of an endoscope. A rotational element can be introduced into the working channel to rotate the springs through the tissue. Use of such a device capable of capturing both tissue mounds simultaneously, will ensure proper spacing between the tissue mounds that are to be joined together. However, the spring devices 390 may be introduced individually through tissue mounds that are captured separately.

Regardless of whether the coil spring devices 390 are delivered separately or together, as shown in FIG. 17B, the springs are joined together in a secondary step by interlacing of individual coils 392 that remain exposed from the tissue. These exposed portions of the springs may be manipulated to come into contact with each other by any conventional means of remote manipulation such as forceps or hemostat, which may be introduced separately from the tissue capture delivery device or may be inserted through a lumen or working channel of that delivery device. After joining of the spring devices 390, the tissue mounds 306 are maintained in close proximity together and are distorted somewhat such that the mound shape is retained.

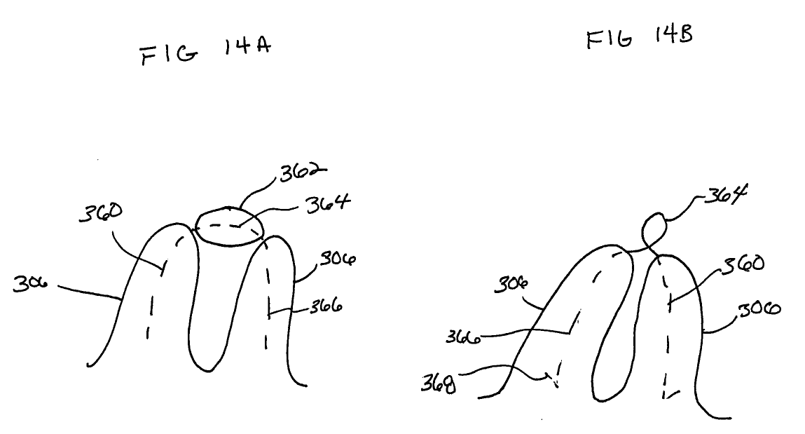

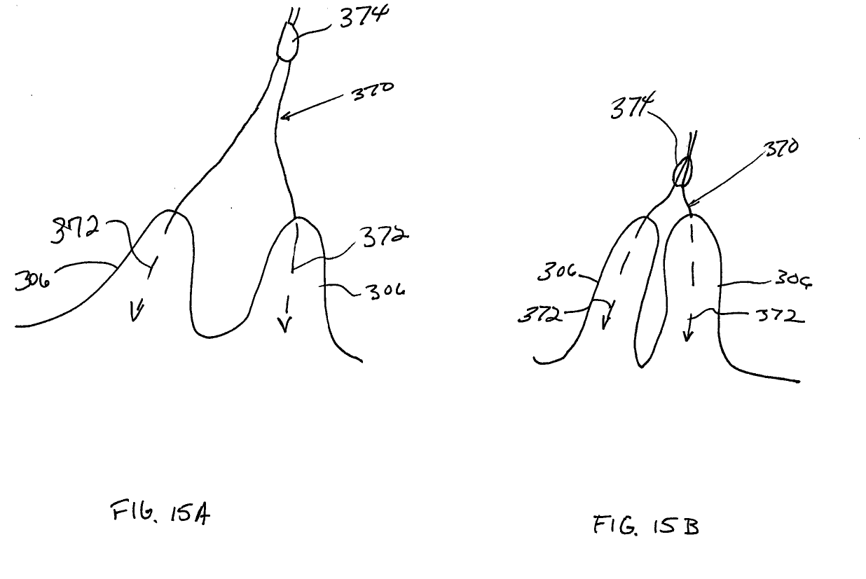

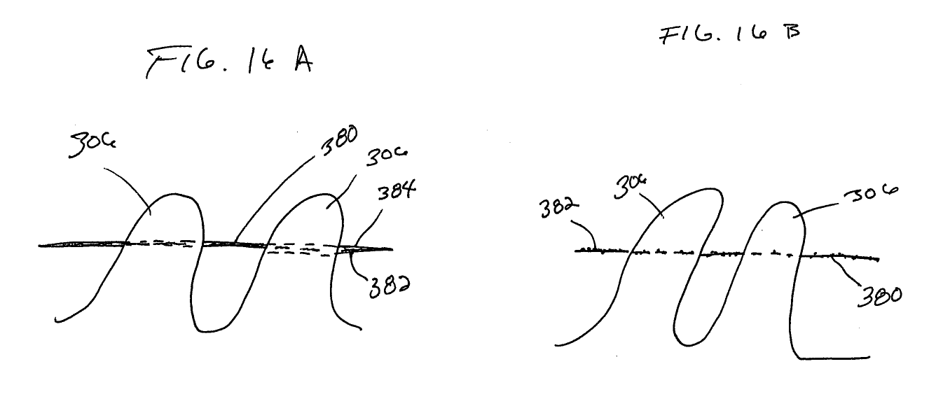

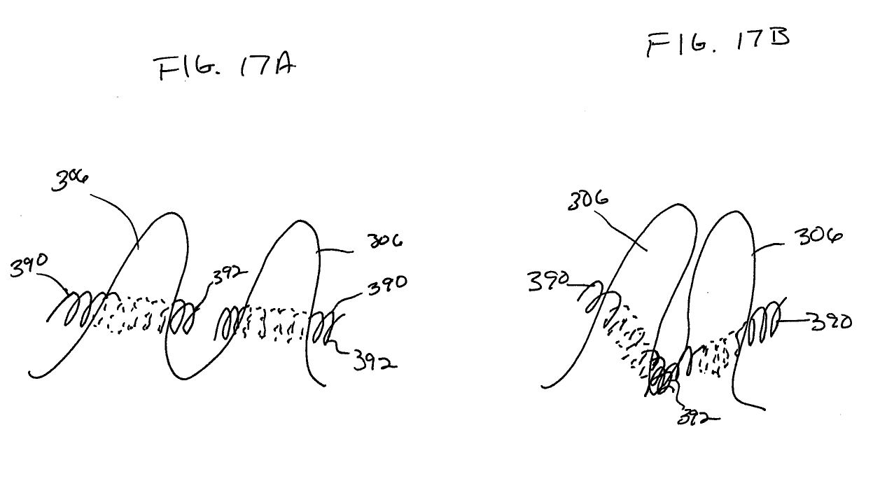

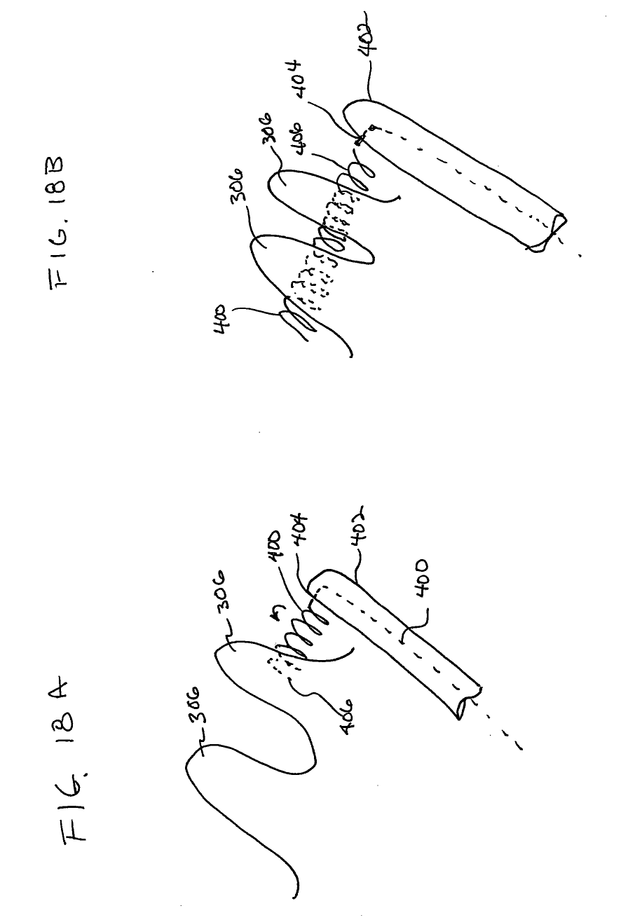

FIG. 18 shows an alternate delivery method for a spring coil type tissue capture device 400. In the delivery method, the spring coil 400 is delivered through the lumen of a catheter or a working channel of an endoscope 402 with the spring in a straightened, uncoiled configuration, shown in FIG. 18A. As the spring coil is pushed through the lumen distally, it emerges through the side port 404 and resumes its coiled configuration forming coils 406 at a right angle to the linear advancement of the straightened portion of the device. As the coils 406 reform, they rotate about an axis that is perpendicular to the linear motion of the straightened portion of the device. The rotating coils penetrate the captured tissue mounds 306 so that the device becomes implanted to capture both mounds in close proximity as shown in FIG. 18B. After the coil 400 has been fully advanced by a longitudinal pusher 408 extending through the lumen of the catheter or endoscope, the device 400 will be shaped entirely of coils 406 to secure the tissue mounds 306 together.

FIGS. 19A-19C show additional tissue capture device embodiments 410, 418 and 424 that are implantable directly into captured tissue mounds and have barbs 412 to prevent the device from becoming withdrawn from the captured tissue portions after implantation. In FIG. 19A the device 410 is provided with multiple barbs 412 spaced along each prong 414 provided for insertion into each captured tissue mound 306. In FIG. 19B a single barb 412 is provided on each prong 420. In FIG. 19C the tissue capture device 424 is provided with a single barb 412 on each prong 422 as with the embodiment described above in connection with FIG. 19B. However, the device 424 further includes a tab 426 serving as a junction for the ends of each prong 412. The tab 426 provides a convenient means for varying the number of prongs 412 that can extend from a given device. In other words, 1wo, three or more tissue mounds could be captured with a single device by providing the necessary number of prongs and joining them together at the tab 426. Additionally, the tab is beneficial in stabilizing the device during implantation. It is noted that each of the embodiments shown in FIGS. 19A-19C may be formed from flexible stainless steel that is resiliently bendable. The devices maintain their shape (generally U-shaped) but may be deflected as required during insertion into the tissue mounds 306. It is noted that the barbs 412 may be deflected to a low profile configuration during insertion into the tissue, but if provided with an arrow shape, they will become anchored within the tissue upon application of a withdrawal force on the device.

FIG. 20 shows a tissue capture device 430 that may be molded as a single element having a linear interior tissue portion 432 that is inserted through pre-captured tissue mounds 306. The device 430 further comprises an external portion 434 configured to loop around the captured tissue mounds 306 and engage the linear interior tissue portion 432 at contact points 436 that remain exterior to the tissue to lock the device 430 in place. The exterior portion 434 may be flexible or semi-rigid and may hook onto the straight portion such as a safety pin may be flexed into a catch to be placed in a locked position at contact points 436.

FIG. 21 shows another embodiment of the tissue capture device that may be inserted through pre-captured tissue portions 306 and lock the portions together without undergoing a configuration change in areas that remain inside the tissue. The device 440 may comprise a single linear element of sufficient length to extend through a desired number of adjacent tissue portions 306. The interior tissue portions 444 remain unchanged after implantation. However, the device 440 is locked in position within the tissue by locking discs 442 applied at the proximal and distal ends of the device where it protrudes from the tissue portions. The device may be applied by a tissue apposition device as shown in FIG. 6, with the linear device being inserted along the longitudinal axis of the device, through the working channel of the endoscope, when the tissue mounds 306 are collected in the suction ports. The proximal blocking disc 442 may be in place already while the linear device is advanced distally such that it is inserted through the distal locking disc 442. The locking disc may comprise a commonly available locking washer having a small center cut out consisting of a hole with several radial slots extending therefrom that serves to lock around a cylinder to prevent sliding motion of the disc relative to the cylinder by virtue of the slotted surfaces of the disc biting into the surface of the cylinder when relative motion is applied. The device in FIG. 20 may also be delivered through the tissue apposition device of FIG. 6 with the external portion 434 disengaged from contact points 436 so that the linear interior portion 432 can be inserted through the captured tissue mounds 306 from the working channel of the endoscope. By secondary device, the external portion 434 may be latched onto the contact points 436 of the device such as an endoscopic forceps device. To facilitate the positioning of the exterior portion 434, it may be pre-attached to the proximal contact point 436 of the device that need not be inserted through a tissue portion.

FIG. 22 shows another embodiment of a tissue capture device delivered into pre-captured tissue mounds that does not require a shape change after delivery to attain the tissue mounds in close proximity to each other. The device 450 comprises a helical coil spring that is wound in two opposing helical directions. A proximal portion of the spring 52 is wound in a first helical direction while the distal portion 454 of the spring is wound in the opposite helical direction so that once implanted in the tissue, each end of the spring will restrain the other end from unwinding out of the tissue. The spring is preferably wound from a flat metal ribbon to provide a greater contact area with the tissue. The ribbon may be canted so that the cross section of each coil 456 presents an angle that is acute to the longitudinal axis of the spring coil 450. To delivery the device, the tissue apposition device as shown in FIG. 6 may be used to pre-capture the multiple tissue mounds 306. The device 450 may be delivered longitudinally through the tissue mounds in a hypotube or hypodermic needle then pushed out of the tubing while placed within the tissue to avoid interference of the reverse wound coils of the device with the tissue during insertion.

FIG. 23 shows another embodiment of the tissue capture device employing a rigid device configured as a dart for penetrating and becoming retained in an area of tissue. The dart 460 is configured to have a penetrating distal tip 462, possible with an arrowhead shape to resist migration from the tissue after implantation. Extending proximally from the arrowhead 462 is a straight stem portion 464 that terminates in a tether receptacle portion 468 having a tether hole 466 for receiving a tether 470 to join the dart 460 to other darts 460 placed in adjacent tissue areas as shown in FIG. 23B. FIG. 23B shows in diagrammatic fashion the placement of several tissue capture darts 460 in adjacent areas of tissue. The multiple darts are joined together by a tether 470, which when pulled tightly through the several darts, gathers the darts together and serves to pull the penetrated tissue areas into mounds 306.

A device for delivering multiple darts to a plurality of tissue areas is shown in FIGS. 24A-24G. The dart delivery device 472 may be similar to the prior art band ligator device shown in FIGS. 7A-7C. The delivery device 472 is configured to be mounted at the distal end of an endoscope 118 as shown in FIG. 24A and comprises a slender pole suction chamber 474 with a supple distal tip 476 for engaging tissue areas and for creating a relatively vacuum tight seal such that when suction is applied to the chamber 474, a tissue mound 306 is drawn into the chamber. The suction chamber also supports along the center of its longitudinal axis a rotatable auger spring 478 for driving the darts distally into the captured tissue mound 306. The spring 478 rotates under motion from torque cable 480 that extends through the working channel of the endoscope 118 and joins the spring in the suction chamber 474. Multiple darts 460 reside between the coils 482 of the spring such that coils fit closely against the stem portion 464 of the dart and abut the enlarged penetrating tip 462 and tether receptacle 468. In this engagement, when the spring rotates, the darts 460 will be advanced as a ride between the individual coils 478. As shown in FIG. 24B, continued rotation of the auger spring 478 serves to drive the first distal dart 460 into the captured tissue mound 306. The darts are pre-loaded with a tether 470 that is not yet tightened so that the darts can be aligned longitudinally in the auger spring for sequential delivery. FIG. 24C shows a dart fully seeded into a tissue mound 306 such that the penetrating tip 462 and stem 464 are embedded in the tissue mound and the tether receptacle 468. After implantation of the first dart, the vacuum is released and the delivery device 472 moved to a new tissue location. As shown in FIG. 24D, a new tissue mound is aspirated into the suction chamber 474 and as shown in FIG. 24E, the auger spring 478 is rotated to advance the second dart 460 into the second tissue mound 306. Tether 470 remains joined to both the first and second darts 460 throughout the delivery process. After delivery of the second dart, the vacuum may be released, leaving the implanted darts 460 in tissue that has returned to its natural configuration. Tether key 482, which has also been advanced in line behind the darts by the rogation of the auger spring 478, receives the free end of the tether 470. After delivery of the second dart 460, the auger spring 478 is rotated and reversed to draw the tether key 482 proximally in order to tighten the tether 470 between the two implanted darts 460 as is shown in FIG. 24F. The tether hole 466 of the tether receptacle 468 of each dart may be configured to receive the tether 470 in a ratcheted fashion such that the tether passes freely in one direction (i.e., the direction of tightening) but is locked and prevented from sliding in the opposite direction (i.e., the direction that loosens the tether between the two darts). Such a ratcheting configuration may be similar to that of the locking disc described in the embodiments of FIG. 21. As shown in FIG. 25, after the tether 470 has been pulled to draw the two implanted darts 460 together, the tissue into which they are implanted again form defined mounds 306 with perhaps some additional folds 484 present between the captured mounds. After the tether has been tightened sufficiently, the tether key 482 can be triggered to release the free end of the tether so that the delivery device 472 can be removed from the tissue location.

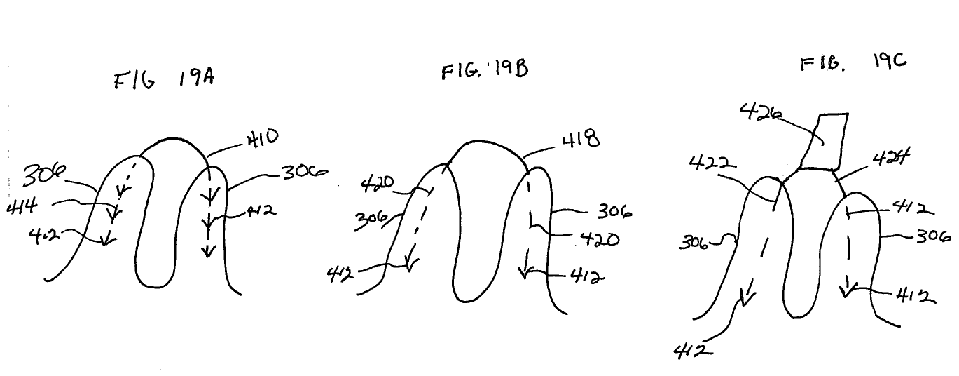

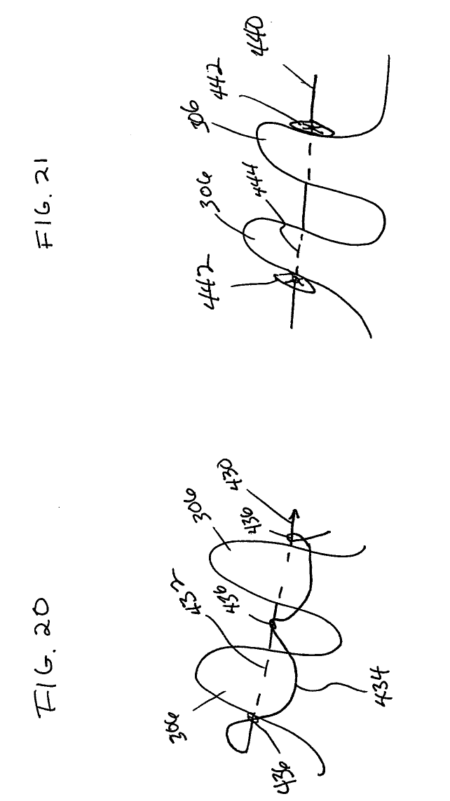

FIG. 26A shows an embodiment of the invention employing a tissue apposition device configured as a band ligator such as that shown in FIGS. 7A-7C discussed above. The bank ligator is advanced to adjacent tissue portions, tissue mound 306 aspirated in bands 134 released on the tissue mounds and endoscopic band ligator instrument removed, shown in FIG. 26B, next, a separate tissue capture delivery device 474 is advanced to the adjacent tissue mounds 306, now defined by ligating bands 134, temporarily placed around them. A tissue capture device 476 comprising a length of filament material and having arrow shaped barbs at each end is then advanced from the delivery device 474 directly into one of the tissue mounds 306 with continued advancement by pusher 478 so that at least one of the barbs 480 from the tissue capture device reaches the adjacent tissue mound 306 as shown in FIG. 26C. With each tissue mound 306 receiving an opposite facing barb 480, the mounds will be held in close proximity. After delivery of the tissue capture device 476, the bands either may be cut away from the tissue portions or may be made of a dissolvable material so that the tissue mounds 306 are left with only the capture device 476 placed to hold them together as shown in FIG. 26D.

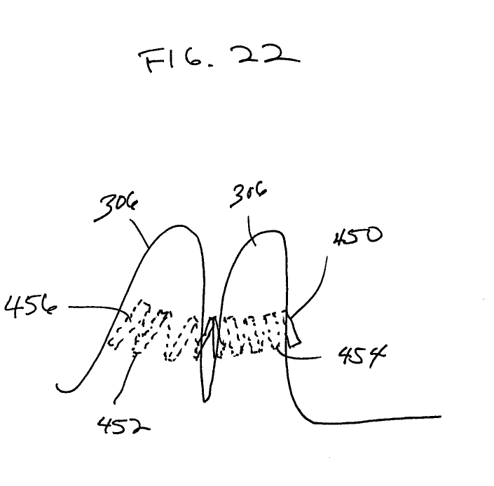

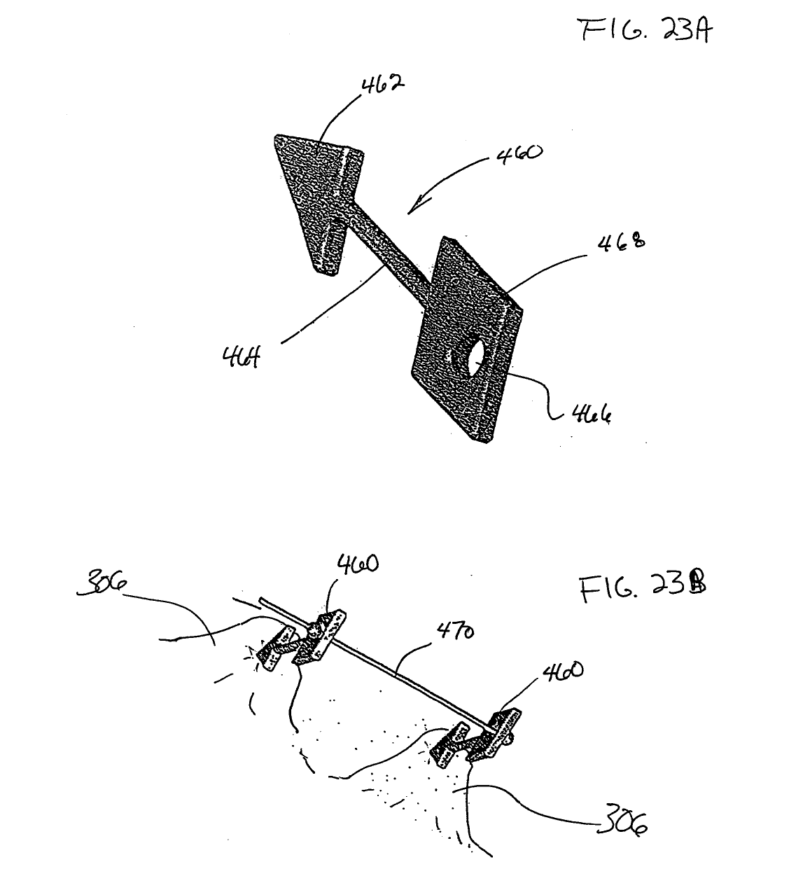

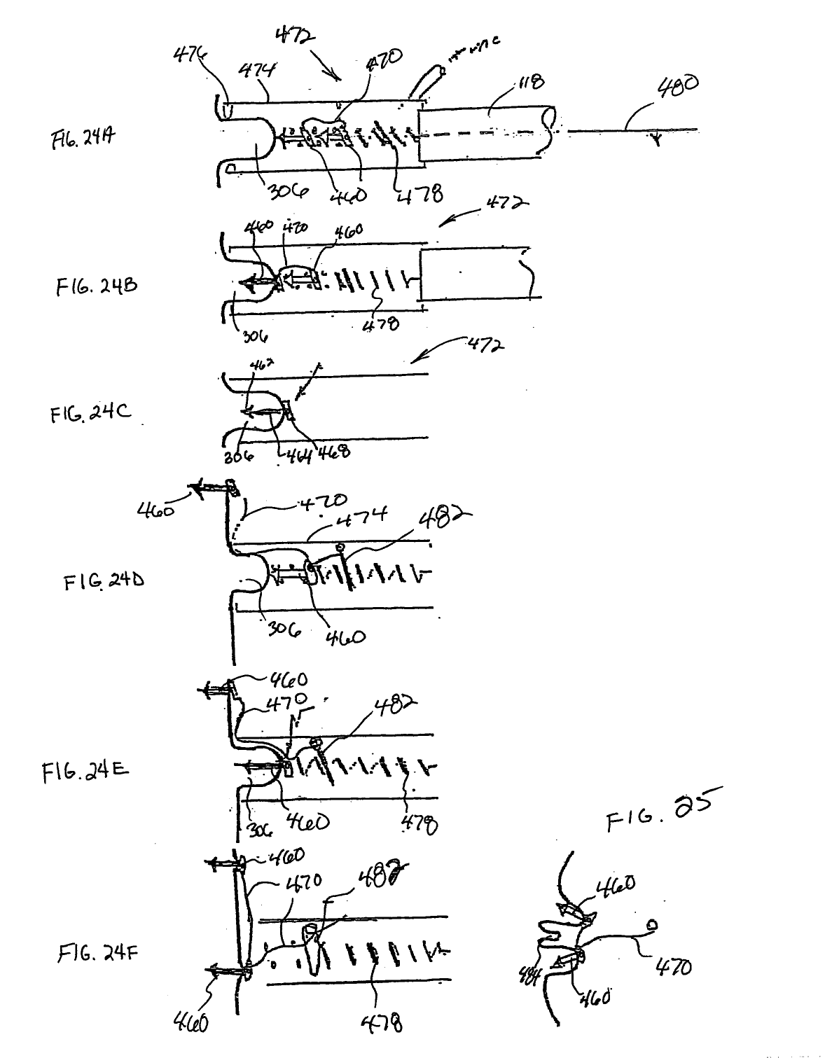

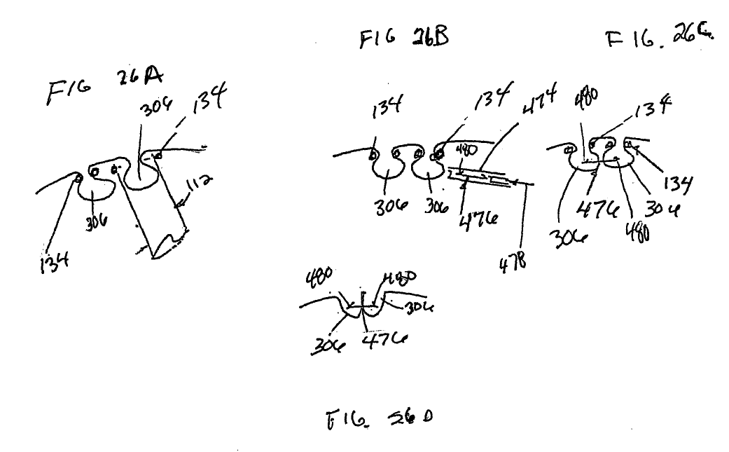

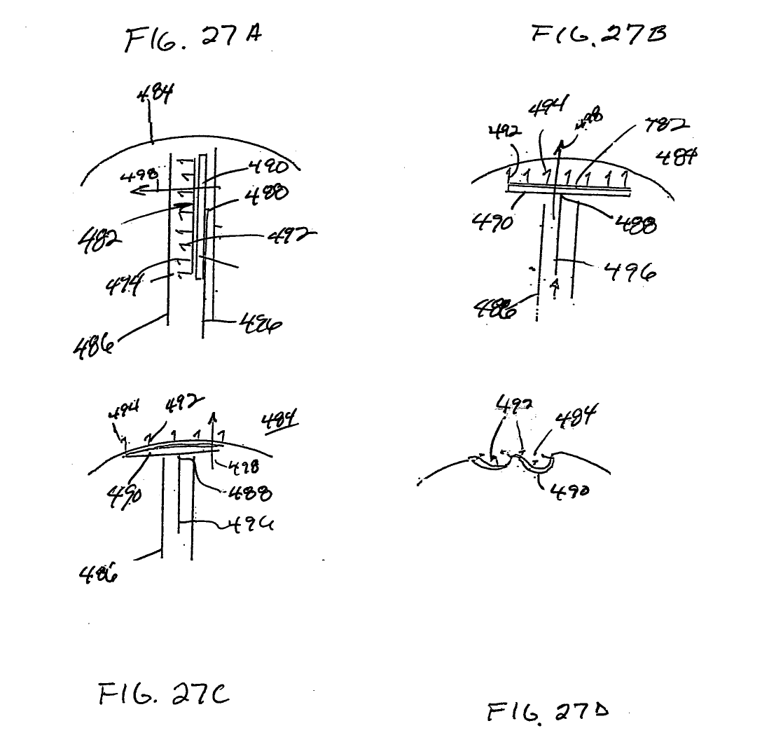

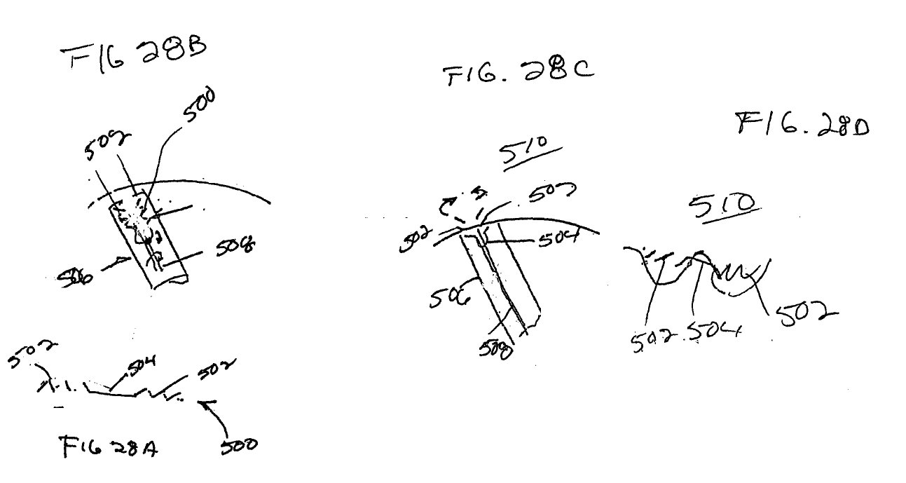

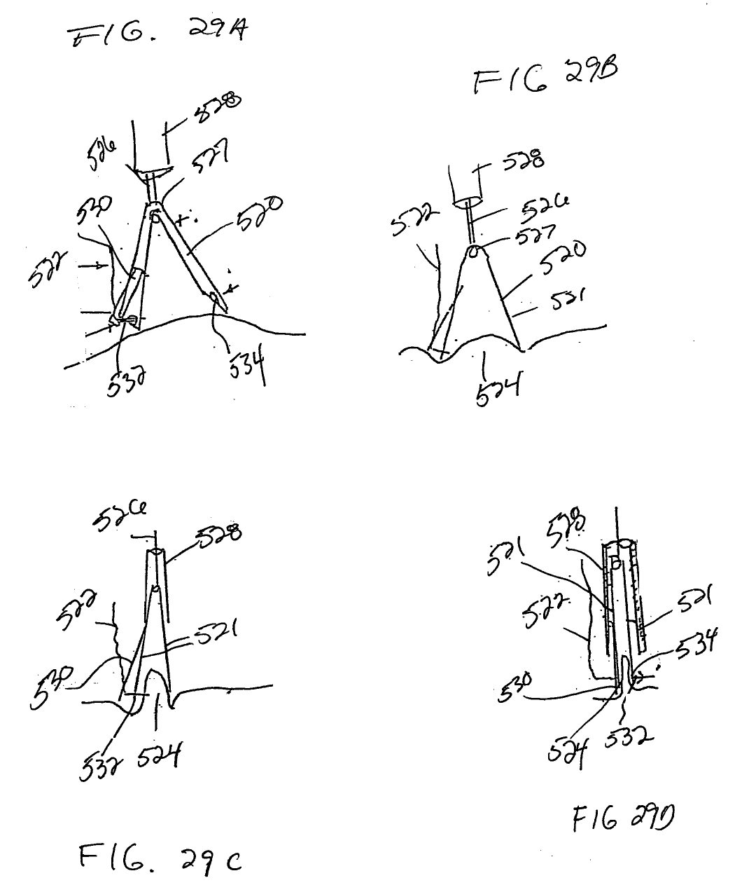

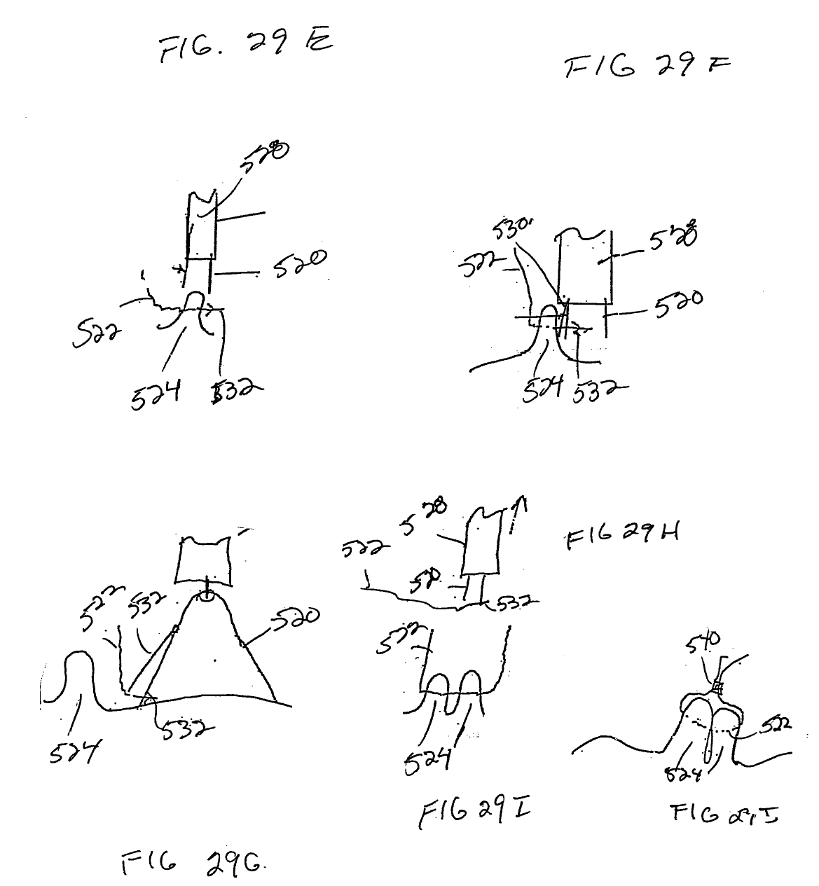

FIGS. 27A-27D show another embodiment of the tissue capture device that may be implanted into tissue that is not pre-deformed by aspiration or a ligating band. The tissue capture device 482 comprises a nitinol substrate base 490 from which projects a plurality of tissue piercing prongs 492 having barbs 494 at their ends. The capture device may be delivered through a catheter or endoscope 486, advanced by a pusher 496 while being arranged laterally to its axis of penetration shown by arrow 498. (See FIG. 27A). The pusher 496 has a swivel connection 488 with the device 482 that permits the advancement through the catheter 486 in the lateral orientation. Once the device 482 is advanced distally past the end of the shaft 486, the swivel point 488 is spring loaded to rotate the device 90° so that its access of deviceation 498 is in align with the longitudinal access of the catheter 486 and pusher 496 so that further distal advancement of the pusher will result in penetration of the barbs 492 into the tissue 484 as shown in FIGS. 27B and 27C. After exposure to the warm internal body temperature, the nitinol base 490 having a shape memory configuration that is non-linear and compacted such as a sinusoidal shape shown in 27D transforms to its stored shape. The new shape of the base 490 causes the tissue captured by prongs 492 to become distorted and follow the shape of the base 490 as shown in FIG. 27D.

Another embodiment of the tissue capture device is shown in FIGS. 28A-28D. FIG. 28A shows a tissue capture device 500 comprising two coil spring segments 502 joined by a nitinol super elastic hypo tube 504. The super elastic hypo tube permits the device to be folded in half and advance through a catheter or endoscope 506, as shown in FIG. 28B, with the spring portions 502 leading distally and in parallel through the scope 506. The hypo tube, positioned proximally within the lumen on the endoscope 506 is engaged by a rotational pusher 508 that engages the hypo tube 504 and uses it as a universal joint to in part rotation to both coil spring as segments 502. As the rotational pusher 508 advances distally, it imparts a rotation to the continuously bending hypo tube 504. The axis of rotation of the hypo tube 504 is parallel to the drawing page. The resulting spinning motion of the coils 502 permits them to drive into the tissue 510 as two cork screws as shown in FIG. 28C once the coil springs have fully embedded in the tissue 510, the pusher 508 may be disengaged from the hypo tube 504 and the endoscope 506 removed, when the capture devise is released, it will resiliently return to a relatively straight shape as shown in FIG. 28D. The resulting deformation of the tissue causes two distinct mounds as shown in FIG. 28D.

Another embodiment of the tissue capture device is presented in FIGS. 29A-29J. In this embodiment, the capture device is a resiliently opened V shaped apparatus configured similar to tweezers. The tweezer device 520 temporarily captures tissue to deliver a suture 522 through the collected tissue portion 524. The tweezer 520 is advanced through a sleeve 528 (FIG. 29C) by a push rod 526 joined to the apex 527 of the tweezer 520. When the tweezer is advanced out of the sleeve 528, it resiliently opens to its expanded configuration, ready to grasp tissue as shown in FIG. 29B, after the tweezer has been advanced into tissue area 524, the sleeve 528 is advanced over the tweezer apex as shown in FIG. 29C, which forces the tweezer prongs 521 to close and capture a tissue area 524 between them.

As seen in FIG. 29D, after the sleeve 528 has been advanced over the tweezer 520 a secondary arm 530 carrying a needle 532 advances along the arcing path of one of the tweezer legs 521 to advance the needle 532 through the captured tissue 524. The needle becomes captured on a receiving notch 534 on the opposite tweezer arm 521. Withdrawal of the sheath 528 relative to the tweezers at this point would permit the tweezers to open and notch would pull the needle through the tissue so that it would be withdrawn from the area drawing the suture 522 through the tissue to complete the stitch. However, if an additional stitch is desired to be made, the needle can be left in place through the tissue 524 as shown in FIG. 29E and the device withdrawn from the tissue portion and adjusted so that the secondary arm 530 is brought into contact with the projecting needle 532, engaging it and setting in readiness for another stitch as shown in FIG. 29F. With the needle received in the secondary arm 532, the tweezers are located to a new tissue area and the process described above is repeated to close the tweezers and capture a second tissue portion 524 as is shown in 29G. After capturing the second tissue portion and delivering the needle there through as described above, the device is withdrawn as shown in FIG. 29H carrying the needle and suture 522 leaving the threaded suture 522 through both tissue portions 524 as shown in FIG. 29I. With both suture leads now withdrawn proximally outside the body a suture lock device 540 may be threaded down to the location and advanced to pull the tissue tight and lock it in position to define the tissue portion 524, as is shown in FIG. 29J.

It should be understood however, that the foregoing description of the invention is intended merely to be illustrative thereof and that other modifications, embodiments and equivalents may be apparent to those who are skilled in the art without departing from its spirit.

Having thus described the invention what we desire to claim and secure by Letters Patent is:

1. A tissue capturing element comprising:
an internal tissue contacting portion,
an external tissue portion, and
a tissue contacting anchor for maintaining the element in position relative to the tissue.

2. A tissue capturing element as defined in claim 1 further comprising a first configuration and a second configuration.

3. A tissue capturing element as defined in claim 2 wherein the interior tissue portion is configured to undergo a shape change that distinguishes the second configuration from the first configuration.

4. A tissue capturing element as defined in claim 2 wherein the exterior tissue portion is configured to undergo a shape change that distinguishes the second configuration from the first configuration.

5. A tissue capturing element as defined in claim 2 wherein the tissue anchor is configured to undergo a shape change that distinguishes a second configuration from the first configuration.

6. A method of capturing tissue in a distorted form comprising:
providing a tissue capturing element having an exterior tissue portion, an interior tissue portion and a tissue anchor portion;
inserting the tissue capturing element so that the interior tissue portion is contained within the subject tissue;
deforming the interior tissue portion of the device to engage the tissue.

7. A method of capturing tissue in a distorted form comprising:
providing a tissue capturing element having an exterior tissue portion, an interior tissue portion and a tissue anchor portion;
inserting the tissue capturing element so that the interior tissue portion is contained within the subject tissue;
deforming the exterior tissue portion to engage the tissue.

8. A method of capturing tissue in a distorted form comprising:
providing a tissue capturing element having an exterior tissue portion, an interior tissue portion and a tissue anchor portion;
inserting the tissue capturing element so that the interior tissue portion is contained within the subject tissue;
deforming the tissue anchor portion in order to engage the tissue.

9. A tissue capturing element comprising:
at least one tissue engaging portion;
at least one distortion portion for becoming distorted to hold tissue in a distorted shape; and
at least one securement mechanism for retaining the element in its tissue distorting form.
